(19) United States
(12) Patent Application Publication
(10) Pub. No.: US 2017/0281578 A1
(43) Pub. Date: Oct. 5, 2017

(54) METHODS FOR TREATING METABOLIC DISTURBANCES

(71) Applicant: THE BOARD OF REGENTS OF THE UNIVERSITY OF TEXAS SYSTEM, Austin, TX (US)

(72) Inventors: Charles Y.C. Pak, Dallas, TX (US); Wanpen Vongpatanasin, Dallas, TX (US); Orson W. Moe, Dallas, TX (US)

(21) Appl. No.: 15/467,040

(22) Filed: Mar. 23, 2017

Related U.S. Application Data
(60) Provisional application No. 62/316,800, filed on Apr. 1, 2016.

(51) Int. Cl. A61K 31/194; A61K 9/20; A61K 9/00

(52) U.S. Cl. CPC A61K 31/194; A61K 9/0053; A61K 9/0095; A61K 9/20

(57) ABSTRACT

In some aspects, the present invention provides a composition comprising a powder or mixture comprising from about 10 meq to 50 meq of potassium, 5 meq to 25 meq of magnesium, and from about 18 meq to 90 meq of citrate and uses of the same to prevent or treat metabolic disturbances of thiazide or chlorthalidone.

### PRIORITY CLAIM

This application claims priority to U.S. Application No. 62/316,800 filed Apr. 1, 2016, which is incorporated herein by reference in its entirety.

### BACKGROUND OF THE INVENTION

### I. Field of the Invention

The present invention relates generally to the fields of biology, chemistry, and medicine. More particularly, it concerns methods and compositions relating to treatments of metabolic disturbances that occur during thiazide treatment such as thiazide treatments for patients with hypertension.

### II. Background

Hypertension, estimated to affect about one-third of the population, is a major cause of morbidity and mortality. Hypertension leads to heart attacks, stroke, congestive heart failure, and renal failure.

For the management of patients with hypertension, the first line treatment is a thiazide (TZ) diuretic, such as hydrochlorothiazide (HCTZ) or related product chlorthalidone (CTD). TZ is widely used; 47.5 million prescriptions were written in 2008 alone.

Unfortunately, TZ treatment is associated with frequent metabolic complications. Noting increased occurrence of insulin resistance and dyslipidemia during TZ treatment of hypertension, it has been said that “[h]ypertensive individuals at risk for diabetes and those with hepatic steatosis should opt for antihypertensive agents that lower blood pressure without exaccerbating patient's metabolic profile” (Price, 2013).

TZ has been shown to cause various metabolic disturbances, some of which are associated with Type II diabetes. These disturbances include hypokalemia, activation of renin angiotenin aldosterone—sympathetic nervous system (RAA-SNS), oxidative stress, dylipidemia, enhanced FGF23 synthesis, insulin resistance, and magnesium depletion. Potassium chloride (KCl) supplementation is approved and indicated for the prevention or correction of hypokalemia from TZ therapy. However, KCl does not ameliorate other metabolic complications cited above. Therefore, there is a need in the art to prevent or ameliorate thiazide-induced metabolic disturbances.

### SUMMARY OF THE INVENTION

The current disclosure fulfills the aforementioned need in the art by providing compositions that are useful for the amelioration of metabolic disturbances in a patient on TZ diuretic therapy. Certain aspects relate to a method of preventing or treating metabolic disturbances in patients receiving or prescribed a TZ diuretic therapy, the method comprising administrating a composition comprising from about 10 meq to 50 meq of potassium, 5 meq to 25 meq magnesium, and from about 18 meq to 90 meq of citrate per dose.

In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meq of potassium, or any range derivable therein. In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, or 25 meq of magnesium, or any range derivable therein. In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 meq of citrate, or any range derivable therein.

In some embodiments, the composition comprises from about 10 meq to 50 meq of potassium citrate, from about 5 meq to 25 meq of magnesium citrate, and 4 meq to 40 meq citric acid. In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 meq of potassium citrate (or any range derivable therein). In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, or 25 meq of magnesium citrate (or any range derivable therein). In some embodiments, the composition comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 meq of citric acid (or any range derivable therein).

In some embodiments, the ratio of potassium to magnesium (expressed in milliequivalents) is at least, at most, or exactly 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0 to at least, at most, or exactly 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0 (or any derivable range therein). In some embodiments, the ratio of potassium to citrate is at least, at most, or exactly 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0 to at least, at most, or exactly 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, or 8.0 (or any derivable range therein). In some embodiments, the ratio of magnesium to citrate is at least, at most, or exactly 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, or 5.0 to at least, at most, or exactly 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, or 5.0 (or any derivable range therein). In some embodiments, the ratio of K/Mg is 2. In some embodiments, the ratio of K/citrate is 0.54. In some embodiments, the ratio of Mg/citrate is 0.27. In some embodiments, the ratio of K/Mg is 2, the ratio of K/citrate is 0.54, and the ratio of Mg/citrate is 0.27.

In some embodiments, the composition comprises KMgCit with 40 meq K, 20 meq Mg and 74 meq citrate powder. In some embodiments, a composition comprising 40 meq K, 20 meq Mg and 74 meq citrate powder is administered in two divided doses twice daily after dissolution in an aqueous medium.

The composition may be in any appropriate form. In some embodiments, the mixture is in the form of a tablet. In some embodiments, the mixture is in the form of a powder. In some embodiments, the tablet is a pressed combination of the powder.

The potassium may be provided in any appropriate form. In some embodiments, the potassium is potassium citrate, potassium carbonate, potassium bicarbonate, or potassium acetate. The magnesium is magnesium citrate, magnesium acetate, magnesium oxide, magnesium hydroxide, or magnesium carbonate. The citrate may be provided in any appropriate form. In some embodiments, the citrate is potassium citrate, citric acid, or magnesium citrate. In some embodiments, the potassium and citrate are potassium citrate. The magnesium and citrate are magnesium citrate. In some embodiments, the composition further comprises additional citric acid or a taste enhancer to improve the taste.

In some embodiments, the composition comprises 20 meq potassium, 10 meq magnesium, and 37 meq citrate. In some embodiments, the composition consists essentially of 20 meq potassium, 10 meq magnesium, and 37 meq citrate. In some embodiments, the composition consists of 20 meq of potassium, 10 meq magnesium, and 37 meq of citrate.

In some embodiments, the patient has hypertension. In some embodiments, the hypertension is essential hypertension. In some embodiments, the TZ diuretic is HCTZ or CTD. In some embodiments, the TZ is CTD. In some embodiments, the TZ diuretic is one ore more TZ diuretics described herein. In some embodiments, the methods exclude one or more TZ diuretics described herein.

In some embodiments of the disclosure, administration of hydrochlorothiazide is specifically excluded in the methods described herein. Therefore, in some embodiments, the thiazide is not hydrochlorothiazide.

The administration of the composition may precede or follow the TZ therapy by intervals ranging from minutes to weeks. In embodiments where the composition and TZ therapy are administered separately, one would generally ensure that a significant period of time did not expire between the time of each delivery, such that the therapeutic composition would still be able to exert an advantageously combined effect on the subject. In some embodiments, it is contemplated that one may administer both modalities within about 4-24 h of each other or within about 4-12 h of each other. In some embodiments, the time between administration between the TZ and composition may be 1, 2, 3, 4, 5, 6, 7 days, or any derivable range therein. In some embodiments, the TZ is administered before the composition of the disclosure. In some embodiments, the composition of the disclosure is administered before the TZ therapy. In some embodiments, the two treatments are concurrent.

Various combinations of therapy may be employed, for example if TZ therapy is labeled “A” and a composition of the disclosure is labeled “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the compositions of the disclosure to a patient/subject will follow general protocols for the administration of such compounds, taking into account the toxicity, if any, of the composition. It is expected that the treatment cycles would be repeated as necessary. It is also contemplated that various standard therapies, such as other antihypertensive agents, may be applied in combination with the described therapy.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults and juveniles. In some embodiments, the patient is a human.

In some embodiments, the subject is taking potassium supplement to treat hypokalemia from TZ. In some embodiments, the patient is one that has been diagnosed with a metabolic disturbance. In some embodiments, the metabolic disturbance is a TZ-induced metabolic disturbance. In some embodiments, the metabolic disturbance is hypokalemia, activation of renin angiotensin aldosterone system and sympathetic nervous system (RAA-SNS), oxidative stress, dyslipidemia, increased FGF23 synthesis, insulin resistance, or Mg depletion. In some embodiments, the subject with hypertension taking TZ suffers from other metabolic disturbances, such as activation of RAA-SNS, oxidative stress, dylipidemia, enhanced FGF23 synthesis, insulin resistance, and magnesium depletion.

The composition may be administered in any suitable manner. For example, it may be administered systemically, orally, via infusion, via continuous infusion, via a lavage, in cremes, or by other method or any combination of the foregoing as would be known to one of ordinary skill in the art (see, for example, Remington's Pharmaceutical Sciences, 1990). In some embodiments, administration comprises oral administration.

The composition may be administered to (or taken by) the patient 1, 2, 3, 4, 5, or 6 times, or any range derivable therein, and they may be administered every 1, 2, 3, 4, 5, 6, 7, 8, 9, 10, 11, 12, 18, or 24 hours, or 1, 2, 3, 4, 5, 6, or 7 days. It is specifically contemplated that the composition may be administered once daily, twice daily, three times daily, four times daily, five times daily, or six times daily (or any range derivable therein) and/or as needed to the patient. In some embodiments, the composition is administered two, three, or four times per day. Alternatively, the composition may be administered every 2 to 24 hours (or any range derivable therein) to or by the patient. It is specifically contemplated that the composition may be administered daily over the course of multiple months or years, such as 1, 2, 3, 4, 5, 6, 7, 8, 9, 10, 11 months or 1, 2, 3, 4, 5, 6, 7, 8, 9, 10, 15, 20, or 30 years (or any derivable range therein) or for an indefinite period of time. The compositions may be administered one or more times in such daily adminstration. In some embodiments, the compositions are administered 1 to 10 times or more.

In some embodiments, the powder or mixture is dissolved in water or aqueous media before oral ingestion. In some embodiments, the powder or mixture is dissolved in water at a time period of less than 6, 5, 4, 3, 2, 1, hours or 50, 40, 30, 20, 10, 5, or 1 minutes before administration (or any derivable range therein). In some embodiments, the composition is further defined as an aqueous solution. In some embodiments, the method may further comprise preparing the aqueous solution by dissolving the dose comprising the mixture in water. In some embodiments, the powder or mixture is to be added to food before oral ingestion. In some embodiments, the composition is in the form of a tablet.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

Methods may be used in combination with TZ diuretic employed as antihypertensive therapy. In some embodiments, potassium magnesium citrate as embodied by this invention replaces potassium chloride supplementation given to prevent hypokalemia from TZ treatment. Potassium magnesium citrate may also be combined with other antihypertensive drugs in patients who display metabolic disturbances of TZ. Combination therapy may be achieved by use of a single pharmaceutical composition that includes both agents, or by administering two distinct compositions at the same time, wherein one composition includes the distinct pharmacological measures and the other includes the second agent(s).

“Effective amount” or “therapeutically effective amount” or “pharmaceutically effective amount” means that amount which, when administered to a subject or patient for treating a disease, is sufficient to effect such treatment for the disease. In some embodiments, the subject (for a 70 kg human being), is administered a dose of 10 meq to 100 meq of potassium, 5 meq to 50 meq magnesium, and 20-180 meq citrate/day. In some embodiments, the effective amount is the above said amounts given in divided doses.

The term “effective,” as that term is used in the specification and/or claims, means adequate to accomplish a desired, expected, or intended result.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the term “water soluble” means that the composition dissolves in water at least to the extent classified as soluble according to literature precedence, to yield at least 100 meq of potassium and 50 meq magnesium per 6 ounces (glassful) of water.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the claims. Embodiments are contemplated wherein a feature or embodiment described herein is specifically excluded from the invention.

All of the methods and apparatuses disclosed and claimed herein can be made and executed without undue experimentation in light of the present disclosure. While the compositions and methods of this invention have been described in terms of preferred embodiments, it will be apparent to those of skill in the art that variations may be applied to the methods and apparatuses and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows changes in serum highly sensitive C-reactive protein (hsCRP) (mcg/ml) in 16 untreated hypertensive subjects at baseline, after CTD treatment for 12 weeks, and after spironolactone (Spiro) treatment for 12 weeks. Serum hsCRP rose significantly from 3.2 mcg/ml (1.0-4.6; median, 25th-75th percentile) at baseline to 4.8 mcg/ml (2.6-9.8) during CTD (p<0.05). In contrast, Spiro had no effect on hsCRP levels in the same subjects. The results indicated that TZ induced inflammatory changes.

FIG. 2 demonstrates direct evidence of KMgCit powder's inhibition of oxidative stress. Thirty patients with pre- or Stage I hypertension underwent a crossover trial, whereby they took KMgCit powder, KCit (potassium citrate) powder, KCl powder, or placebo for 4 weeks. The potassium content during the K salt phases was 20 meq bid. In the KMgCit powder phase, patients also took 10 meq Mg bid. A spot urine sample was obtained at the end of 4 weeks of treatment of each phase. Spot urinary 8-isoprostane was lower during the KMgCit powder phase than during other phases (** p<0.05 and †<0.001 from the KMgCit powder; p=0.11 between KCit powder and placebo).

FIG. 3 shows that dietary Mg supplementation reduced muscle oxidative stress both in contracting muscle and resting muscle in spontaneously hypertensive rats. An increase in oxidative stress is demonstrated and is evidenced by increased ethidium fluorescence (DHE/DAPI ratio—y axis) in resting and contracting muscles of SHRs treated with Mg deficient diet compared to high Mg diet.

FIG. 4 illustrates effect of KMgCit on serum FGF23. In 30 patients with pre- and Stage I hypertension participating in a crossover randomized trial, serum FGF23 was significantly lower when they were taking KMgCit powder than KCl powder (46.1±16.7 Rel U/ml vs. 53.1±22.2 Rel U/ml, p=0.05).

FIG. 5 illustrates an overall scheme for the development of metabolic disturbances during TZ therapy, and their correction by KMgCit. TZ (CTD or HCTZ) causes hypokalemia, RAA-SNS activation, oxidative stresss, dyslipidemia, enhanced FGF23 synthesis, insulin resistance and magnesium depletion. Some of these disturbances may result in Type II diabetes and metabolic syndrome. KMgCit might ameliorate these disturbances, whereas KCl might only correct hypokalemia.

### DETAILED DESCRIPTION OF THE INVENTION

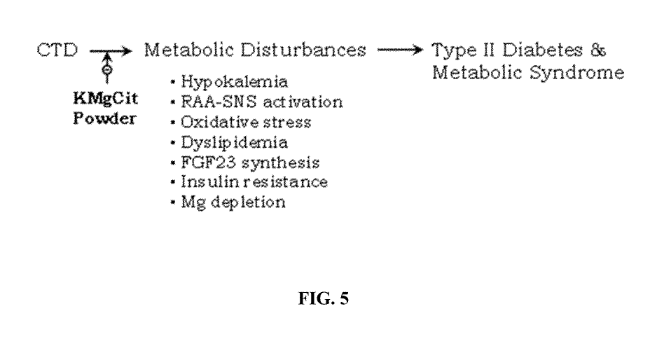

Disclosed is a novel use of potassium magnesium citrate to avert metabolic disturbances of TZ diuretics. Thiazide diuretics (including HCTZ and CTD) are widely used for the management of hypertension as a first line treatment. While it is generally effective in lowering blood pressure, its use is often complicated by many metabolic disturbances, some of which are recognized to be a part of metabolic syndrome or type II diabetes. Potassium chloride supplementation is an accepted co-treatment with TZ, since it can overcome TZ-induced hypokalemia. However, it is ineffective in correcting other metabolic disturbances. In addition to hypokalemia, CTD may produce other metabolic disturbances, including activation of RAA-SNS, oxidative stress, dyslipidemia, FGF23 synthesis, insulin resistance, and magnesium depletion (FIG. 5). These factors act solely or interact with each other to contribute to the development of Type II diabetes and metabolic syndrome. KMgCit powder can potentially overcome all of these metabolic disturbances. Without being bound to any theory, it is believed that co-administration of KMgCit powder would avert Mg depletion, block hepatic fat deposition by restoring normal Mg status and direct intestinal binding of fat, thereby ameliorating insulin resistance.

### A. Metabolic Disturbances of TZ Diuretics

CTD is a thiazide-type diuretic advocated by many guidelines for the treatment of hypertension (NICE 2011), owing to its longer half-life and greater antihypertensive efficacy at the clinically recommended doses than other TZ diuretics (Vongpatanasin, 2015). Despite these favorable properties and increasing popularity, CTD and other TZ diuretics are known to cause various metabolic disturbances, such as hypokalemia, activation of RAA-SNS (Menon, 2009), oxidative stress (Ribeiro, 2013; Reungjui, 2007), dyslipidemia (Eriksson, 2008), increased FGF23 synthesis (Pathare, 2012), insulin resistance (Raheja, 2012; Menon, 2009), and Mg depletion (Hollifield, 1987).

Various metabolic disturbances of CTD/TZ might be pathogenetically linked. Without wishing to be bound by theory, some metabolic disturbance might directly cause insulin resistance and Type II diabetes. Other factors might do so indirectly by affecting other metabolic disturbances. Activation of RAA system may contribute to insulin resistance by inhibiting insulin signaling pathway in the adipocytes and skeletal muscle (Wada, 2009). This effect is mediated at least in part by increased oxidative stress (Sowers, 2009).

Mg depletion from TZ may cause renal potassium loss and refractory hypokalemia (Whang, 1977). Activation of RAA system by TZ may depend on Mg status. In normal subjects, serum aldosterone at baseline and after angiotensin II infusion was significantly higher on Mg-deficient diet than on Mg-replete diet (Nadler, 1993). Co-infusion of Mg with angiotensin II attenuated the rise in serum aldosterone, suggesting direct inhibitory effect of Mg on the RAA system.

### B. KMgCit Compositions

Effective strategy in preventing the metabolic disturbances of TZ has not been fully developed. Correction of hypokalemia by KCl did not reverse CTD-induced fasting hyperglycemia and increased HOMA-IR (Raheja, 2012; Menon, 2009). CTD-induced activation of RAA-SNS was unaffected by KCl supplementation (Menon 2009, Raheja 2012). In TZ-treated patients with hypertension, KCl supplementation did not reduce serum aldosterone (Kaplan, 1985).

KMgCit possesses distinct properties apart from KCl powder in overcoming deleterious metabolic disturbances of TZ therapy.

Physiological properties of KMgCit. This preparation was shown to confer equivalent potassium bioavailability as potassium chloride, similar magnesium bioavailability as magnesium citrate, and greater alkali load than potassium citrate (Wuermser, 2000; Koenig, 1991; Odvina, 2006; Ruml, 1999). Some of the orally administered citrate may appear in urine by escaping hepatic metabolism in vivo, contributing to the rise in serum citrate. The above actions of KMgCit—potassium and magnesium load, citraturic action, and alkali load—have beneficial roles in mitigating metabolic disturbances of TZ.

The compositions described herein may be administered to a subject in need of treatment by a variety of routes of administration, including orally and parenterally (e.g., intravenously), as a suppository or using a “flash” formulation, i.e., allowing the medication to dissolve in the mouth without the need to use water, topically, and/or administration via mucosal routes in liquid or solid form. The composition can be formulated into a variety of dosage forms, e.g., extract, pills, tablets, microparticles, capsules, powder in sachet or packets, or oral liquid.

There may also be included as part of the composition of pharmaceutically compatible binding agents, and/or adjuvant materials. The compositions can also be mixed with other active materials including antibiotics, antifungals, other virucidals and immunostimulants which do not impair the desired action and/or supplement the desired action.

In one embodiment, the mode of administration of the pharmaceutical composition described herein is oral. Oral compositions generally include an inert diluent or an edible carrier. They may be enclosed in gelatin capsules or compressed into tablets. For the purpose of oral therapeutic administration, the aforesaid compounds or agents may be incorporated with excipients and used in the form of tablets, powder in sachet or packets, troches, capsules, elixirs, suspensions, syrups, wafers, chewing gums and the like. Some variation in dosage will necessarily occur, however, depending on the condition of the subject being treated. These preparations should raise serum concentration of active ingredient of from about 0.05 to 0.50 mmol/L potassium, and 0.04 to 0.30 mmol/L of magnesium. In some embodiments, the increment in serum concentration is about 0.3 mmol/L potassium and about 0.1 mmol/L magnesium. However, the concentration of active ingredient in the composition itself depends on bioavailability and other factors known to those of skill in the art.

In another embodiment, the mode of administration of the pharmaceutical compositions described herein is topical or mucosal administration.

Various polymeric and/or non-polymeric materials can be used as adjuvants for enhancing mucoadhesiveness of the pharmaceutical composition disclosed herein. The polymeric material suitable as adjuvants can be natural or synthetic polymers. Representative natural polymers include, for example, starch, chitosan, collagen, sugar, gelatin, pectin, alginate, karya gum, methylcellulose, carboxymethylcellulose, methylethylcellulose, and hydroxypropylcellulose. Representative synthetic polymers include, for example, poly(acrylic acid), tragacanth, poly(methyl vinylether-co-maleic anhydride), poly(ethylene oxide), carbopol, poly(vinyl pyrrolidine), poly(ethylene glycol), poly(vinyl alcohol), poly(hydroxyethylmethylacrylate), and polycarbophil. Other bioadhesive materials available in the art of drug formulation can also be used (see, for example, Bioadhesion--Possibilities and Future Trends, Gurny and Junginger, eds., 1990).

It is to be noted that dosage values also vary with the specific severity of the disease condition to be alleviated. It is to be further understood that for any particular subject, specific dosage regimens should be adjusted to the individual need and the professional judgment of the person administering or supervising the administration of the aforesaid compositions. It is to be further understood that the concentration ranges set forth herein are exemplary only and they do not limit the scope or practice of the disclosure. The active ingredient may be administered at once, or may be divided into a number of smaller doses to be administered at varying intervals of time.

The formulation may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose, sucralose, or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials which modify the physical form of the dosage unit, for example, as coatings. Thus tablets or pills may be coated with sugar, shellac, or other enteric coating agents. Materials used in preparing these various compositions should be pharmaceutically pure and non-toxic in the amounts used.

The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, or citrates and agents for the adjustment of tonicity such as dextrose. The parental preparation can be enclosed in ampoules, disposable syringes or multiple dose vials made of glass or plastic.

The compositions can be prepared as formulations with pharmaceutically acceptable carriers. Preferred are those carriers that will protect the composition against rapid elimination from the body, such as a controlled release formulation, including implants and microencapsulated delivery systems. Biodegradable, biocompatable polymers can be used, such as polyanhydrides, polyglycolic acid, collagen, and polylactic acid. Methods for preparation of such formulations can be readily performed by one skilled in the art.

Liposomal suspensions may also be used as pharmaceutically acceptable carriers. Methods for encapsulation or incorporation of compounds into liposomes are described by Cozzani, I.; Joni, G.; Bertoloni, G.; Milanesi, C.; Sicuro, T. Chem. Biol. Interact. 53, 131-143 (1985) and by Joni, G.; Tomio, L.; Reddi, E.; Rossi, E. Br. J. Cancer 48, 307-309 (1983), for example. These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Jori, G.; Reddi, E.; Cozzani, I.; Tomio, L. Br. J. Cancer, 53(5), 615-21 (1986), for example.

The composition described herein may be administered in single (e.g., once daily) or multiple doses or via constant infusion. The compounds may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this disclosure and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like according to a specific dosage form.

Thus, for example, for purposes of oral administration, tablets containing various excipients such as calcium carbonate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof

For parenteral administration, solutions of the compounds of this disclosure in sesame or peanut oil, aqueous propylene glycol, or in sterile aqueous solutions may be employed.

Such aqueous solutions should be suitably buffered if necessary and the liquid diluent first rendered isotonic with sufficient saline or glucose. These particular aqueous solutions are especially suitable for intravenous, and intraperitoneal administration. In this connection, the sterile aqueous media employed are all readily available by standard techniques known to those skilled in the art.

The pharmaceutical composition provided herein can also be used with another pharmaceutically active agent effective for a disease such as a metabolic disturbance as described herein.

The compositions described herein can be formulated alone or together with the other agent in a single dosage form or in a separate dosage form. Methods of preparing various pharmaceutical formulations with a certain amount of active ingredient are known, or will be apparent in light of this disclosure, to those skilled in this art. For examples of methods of preparing pharmaceutical formulations, see Remington's Pharmaceutical Sciences, Mack Publishing Company, Easton, Pa., 19th Edition (1995).

In some embodiments, the compositions described herein further comprise a carrier. In one embodiment, the carrier may be comprised of sequestering agents such as, but not limited to, collagen, gelatin, hyaluronic acid, alginate, poly(ethylene glycol), alkylcellulose (including hydroxyalkylcellulose), including methylcellulose, ethylcellulose, hydroxyethylcellulose, hydroxypropylcellulose, hydroxypropyl- methylcellulose, and carboxymethylcellulose, blood, fibrin, polyoxyethylene oxide, calcium sulfate hemihydrate, apatites, carboxyvinyl polymer, and poly(vinyl alcohol). See for example, U.S. Pat. No. 6,620,406, herein incorporated by reference.

In one embodiment, the carrier may include buffering agents such as, but not limited to glycine, glutamic acid hydrochloride, guanidine, heparin, glutamic acid hydrochloride, acetic acid, succinic acid, polysorbate, dextran sulfate, sucrose, and amino acids. See for example, U.S. Pat. No. 5,385,887, herein incorporated by reference. In one embodiment, the carrier may include a combination of materials such as those listed above. By way of example, the carrier may be a PLGA/collagen carrier membrane.

In one embodiment, the composition according to this disclosure may be contained within a time release tablet. A bioactive agent described herein can be formulated with an acceptable carrier to form a pharmacological composition. Acceptable carriers can contain a physiologically acceptable compound that acts, for example, to stabilize the composition or to increase or decrease the absorption of the agent. Physiologically acceptable compounds can include, for example, carbohydrates, such as glucose, sucrose, or dextrans, further antioxidants, such as ascorbic acid or glutathione, chelating agents, low molecular weight proteins, compositions that reduce the clearance or hydrolysis of the anti-mitotic agents, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a carrier, including a physiologically acceptable compound depends, for example, on the route of administration.

The composition can have a dosage of about 0.1 g to about 10 g, for example, the dose may be at least, at most, or exactly 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 6, 6.5, 7.0, 7.5, 8.0, 8.5, 9.0, 9.5, or 10.0 g (or any derivable range therein).

Embodiments of the composition can be administered in a variety of unit dosage forms depending upon the method of administration. For example, unit dosage forms suitable may include powder, tablets, pills, capsules.

### C. Thiazide Diuretics

Examples of thiazide diuretics include, without limitation, Hydrochlorothiazide, Chlorothiazide, Losartan/hydrochlorothiazide, Lisinopril/hydrochlorothiazide, Valsartan/hydrochlorothiazide, Co-amilozide, Telmisartan/hydrochlorothiazide, Irbesartan/Hydrochlorothiazide, Aliskiren/amlodipine/hydrochlorothiazide, Triamterene/Hydrochlorothiazide, Methyclothiazide, Bi soprolol/Hydrochlorothiazide, Candesartan/Hydrochlorothiazide, Captopril/Hydrochlorothiazide, Enalapril/Hydrochlorothiazide, Metoprolol/Hydrochlorothiazide, Fosinopril/hydrochlorothiazide, Olmesartan/Hydrochlorothiazide, Benazepril/Hydrochlorothiazide, Spironolactone/Hydrochlorothiazide, Propranolol/Hydrochlorothiazide, Amlodipine/Valsartan/Hydrochlorothiazide, Olmesartan/Amlodipine/Hydrochlorothiazide, Chlorothiazide sodium, Moexipril/Hydrochlorothiazide, Methyldopa/Hydrochlorothiazide, Ali skiren/Hydrochlorothiazide, Eprosartan/Hydrochlorothiazide, Nadolol/B endroflumethiazide, Benazeprilat/Hydrochlorothiazide, Quinapril/Hydrochlorothiazide, Moexiprilat/Hydrochlorothiazide and derivatives or prodrugs thereof.

### EXAMPLES

The following examples are included to demonstrate particular embodiments of the invention. It should be appreciated by those of skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Effect of TZ on Inflammation: Serum hsCRP LEVELS

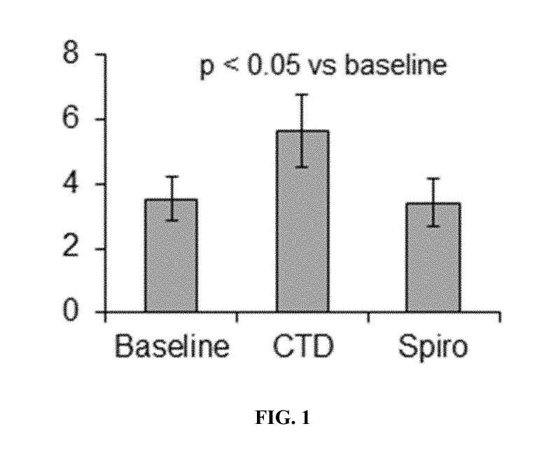

This study illustrates results showing changes in serum highly sensitive C-reactive protein (hsCRP) (mcg/ml) in 16 untreated hypertensive subjects at baseline, after CTD treatment (12.5-25 mg daily) for 12 weeks, and after Spiro treatment (25-50 mg daily) for 12 weeks. The dose of CTD and Spiro was titrated to decrease BP to <140/90 mmHg. Serum hsCRP rose significantly from 3.2 mcg/ml (1.0-4.6; median, 25th-75th percentile) at baseline to 4.8 mcg/ml (2.6-9.8) during CTD (p<0.05). In contrast, Spiro had no effect on hsCRP levels in the same subjects. The results indicated that TZ induced inflammatory changes. These results are shown in FIG. 1.

### Example 2

### Effect of KMgCit on Oxidative Stress: Urinary 8-Isoprostane Significantly Declined on KMgCit Powder

In the inventors' published study (Vongpatanasin, 2016), 30 patients with pre- (6 subjects) or Stage I (24 subjects) hypertension underwent a crossover trial, whereby they took KMgCit powder (20 meq K, 10 meq Mg and 37 meq citrate twice daily), KCit powder (20 meq K and 20 meq citrate twice daily), KCl powder (20 meq K and 20 meq chloride twice daily), or placebo (microcrystalline cellulose) for four weeks each. Among hypertensive subjects, 17 of 24 received antihypertensive treatment before study participation. All treated subjects received single-drug regimen before participation in the study (6 were on β blockers, 11 on angiotensin receptor blockers, or angiotensin-converting enzyme inhibitors, or calcium channel blockers), which was continued at the same dose throughout the study. None of the subjects had low serum Mg<1.8 mg/dl or serum K<3.5 meq/l or were on diuretic therapy.

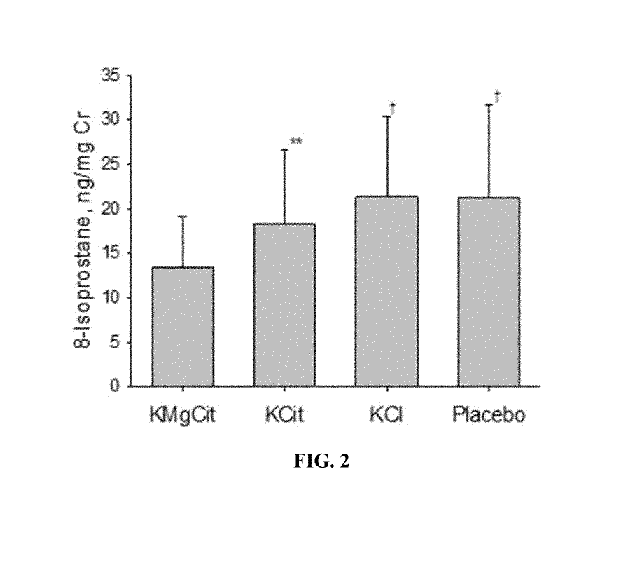

A spot urine sample was obtained at the end of 4 weeks of treatment of each phase. Spot urinary 8-isoprostane was lower during the KMgCit powder phase than during other phases (**p<0.05 and †<0.001 from the KMgCit powder; p=0.11 between KCit powder and placebo). These results are shown in FIG. 2.

Serum K and 24-hour urinary K excretion were increased during KCl, KCit, and KMgCit phases compared with placebo phase (p <0.01 vs placebo). Serum Mg was increased during KMgCit phase compared with placebo and KCl phases (both p<0.01); 24-hour urinary Mg excretion was increased during KMgCit phase compared with placebo and KCit (both p<0.01). 24-hour urinary Mg excretion was higher during KMgCit compared with KCl (p=0.017).

### Example 3

### Effect of MG Deficient and High Mg Diet on Oxidative Stress

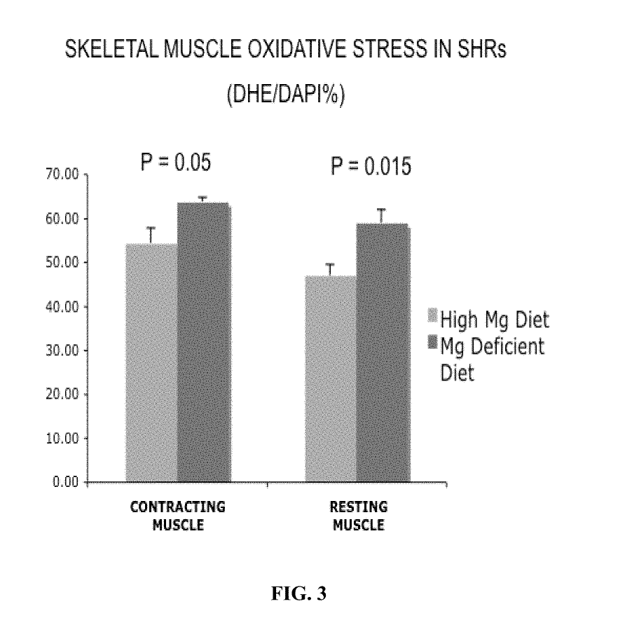

In the inventors' study in spontaneously hypertensive rats (SHRs), dietary Mg supplementation reduced muscle oxidative stress both in contracting muscle and resting muscle. Spontaneously hypertensive rats were evaluated on Mg deficient diet (0.003%, n=5) and high (0.48%, n=5) Mg diet. Summary data shows increased oxidative stress as evidenced by increased ethidium fluorescence (DHE/DAPI ratio) in resting and contracting muscles of SHRs treated with Mg deficient diet compared to high Mg diet. These results are shown in FIG. 3.

### Example 4

### Effect of KMgCit on Serum Triglyceride

In the inventors' prior study (Odvina, & Pak, 2006), previously unpublished data on serum triglyceride were extracted. Eight healthy adult subjects took HCTZ 50 mg/day with KMgCit containing 40 meq K and 20 meq Mg/day for 6 months. Ten subjects received HCTZ 50 mg/day with KC1 40 meq K/day. Serum triglyceride declined by −32±41 mg/dL from 1 month to 5 months of treatment in the KMgCit group; It was underaltered from 1 month to 5 months of KCl treatment (0±31 mg/dL). The difference of the change in serum triglyceride between KMgCit and KCl groups was marginally significant (p=0.09). No baseline or 6 month data were available.

### Example 5

### Effect of KMgCit on Serum FGF23

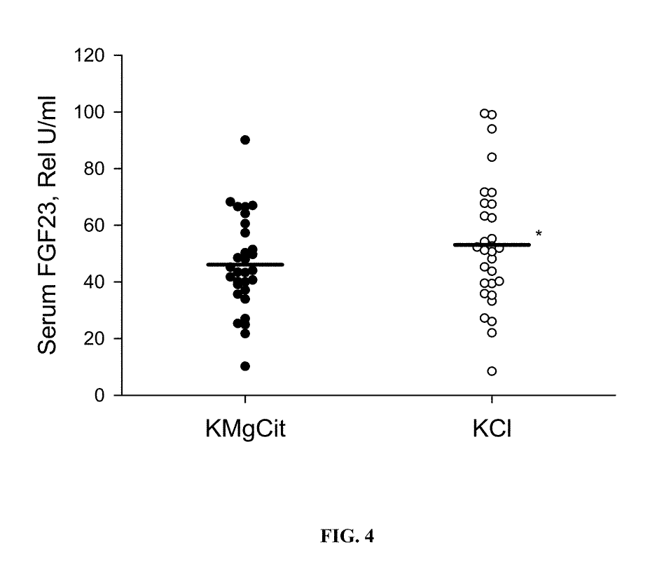

In the inventors' study, 30 patients with pre- or Stage I hypertension participated in a crossover randomized trial, whereby they took KMgCit powder (20 meq K, 10 meq Mg and 37 meq citrate twice daily) or KCl powder (20 meq K and 20 meq chloride twice daily) for four weeks each. After 4 weeks of treatment, serum samples were analyzed fro FGF23. Serum FGF23 was significantly lower when they were taking KMgCit powder than KCl powder (46.1±16.7 Rel U/ml vs. 53.1±22.2 Rel U/ml, p=0.05). These results are shown in FIG. 4.

### Example 6

### Administration of KMgCit Powder

Ordinarily, subjects will dissolve the contents of each packet or sachet of KMgCit powder in about 6 oz water and ingest it with breakfast and again with dinner, to deliver 40 meq K, 20 meq Mg, and 37 meq citrate per day.

A taste enhancer and sucralose will be added to sachet or packet to improve taste and tolerance of KMgCit.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All references cited in this application are specifically incorporated by reference for all purposes

### REFERENCES

The following references, to the extent that they provide exemplary procedural or other details supplementary to those set forth herein, are specifically incorporated herein by reference.

Eriksson J W, Jansson P-A, Carlberg B, et al. Hydrochlorothiazide, but not candesartan, aggravates insulin resistance and causes visceral and hepatic fat accumulation. Hypertension. 52:1030-1037, 2008.

Hollifield J W. Magnesium depletion, diuretics and arrhythmias. Am J Med 82: 30-37, 1987.

Kaplan N M, Carnegie A, Raskin P, et al. Potassium supplementation in hypertensive patients with diuretic-induced hypokalemia. N Eng J Med 312: 746-749, 1985.

Koenig K, Padalino P, Alexandrides G, Pak C Y C. Bioavailability of potassium and magnesium, and citraturic response from potassium-magnesium citrate. J Urol 145: 330-334, 1991.

Menon D V, Arbique D, Wang Z, et al. Differential effects of chlorthalidone versus spironolactone on muscle sympathetic nerve activity in hypertensive patients. J Clin Endo Metab 94: 1361-1366, 2009.

Nadler J L, Buchanan T, Natarajan R, et al. Magnesium deficiency produces insulin resistance and increased thromboxane synthesis. Hypert 21:1024-9, 1993.

NICE, National Clinical Guideline Centre (UK). NICE guidelines [CG127]: The Clinical

Management of Primary Hypertension in Adults: Update of Clinical Guidelines 18 and 34. 2011. http://www.ncbi.nlm.nih.gov/pubmed/22855971.

Odvina C V, Mason R P, Pak C YC. Prevention of thiazide-induced hypokalemia without magnesium depletion by potassium-magnesium citrate. Am J Therap 13: 101-108, 2006.

Pathare G, Föller M, Michael D, et al. Enhanced FGF23 serum concentrations and phosphaturia in gene targeted mice expressing WNK-resistant Spak. Kidn Blood Press Res 36:355-364, 2012.

Price A L, Lingvay I, Szczepaniak E W, Wiebel J, Victor R G, Szczepaniak L S. The metabolic cost of lowering blood pressure with hydrochlorothiazide. Diabet Metab Synd 5:35, 2013.

Raheja P, Price A, Wang Z, et al. Spironolactone prevents chlorthalidone-induced sympathetic activation and insulin resistance in hypertensive patients. Hypertension 60: 319-25, 2012.

Reungjui S, Hu H, Mu W, et al. Thiazide-induced subtle renal injury not observed in states of equivalent hypokalemia. Kid Int 72: 1483-1492, 2007.

Ribeiro M C P, Avila D S, Barbosa N B, et al. Hydrochlorothiazide and high-fat diets reduce plasma magnesium levels and increase levels and increase hepatic oxidative stress in rats. Mag Res 26 (1): 32-40, 2013.

Ruml L A, Pak C Y C. Effect of potassium magnesium citrate on thiazide-induced hypokalemia and magnesium loss. Amer J Kid Dis 34:107-113, 1999.

Sowers J R, Whaley-Connell A, Epstein M. Narrative review: the emerging clinical implications of the role of aldosterone in the metabolic syndrome and resistant hypertension. Ann Inter Med 150(11):776-83, 2009.

Vongpatanasin W. Hydrochlorothiazide is not the most useful nor versatile thiazide diuretic. Curr Opin Cardiol 30(4):361-365, 2015.

Vongpatanasin W, Moe O W, Pak C Y C, et al. Effects of potassium magnesium citrate supplementation on 24-hour ambulatory blood pressure and oxidative stress marker in prehypertensive and hypertensive subjects. Am J Cardiol 118(6):849-53, 2016.

Wada T, Ohshima S, Fujisawa E, et al. Aldosterone inhibits insulin-induced glucose uptake by degradation of insulin receptor substrate (IRS) 1 and IRS2 via a reactive oxygen species-mediated pathway in 3T3-L1 adipocytes. Endocrinol 150:1662-9, 2009.

Whang R, Aikawa J K. Magnesium deficiency and refractoriness to potassium repletion. J Chron Dis 30: 65-68. 1977.

Wuermser L A, Reilly C, Poindexter J R, Sakhaee K, Pak C Y C. Potassium-magnesium citrate versus potassium chloride in thiazide-induced hypokalemia. Kid Int 57: 607-612, 2000.

What is claimed is:

1. A method of preventing or treating metabolic disturbances in patients receiving or prescribed a thiazide diuretic therapy, the method comprising administrating a composition comprising from about 10 meq to 50 meq of potassium, 5 meq to 25 meq magnesium, and from about 18 meq to 90 meq of citrate per dose.

2. The method of claim 1, wherein the potassium is potassium citrate, potassium carbonate, potassium bicarbonate, or potassium acetate.

3. The method of claim 1, wherein the magnesium is as magnesium citrate, magnesium acetate, magnesium carbonate, or magnesium oxide or hydroxide.

4. The method of claim 1, wherein the citrate is potassium citrate, citric acid, or magnesium citrate.

5. The method of claim 1, wherein the composition comprises 20 meq potassium, 10 meq magnesium, and 37 meq citrate per dose.

6. The method of claim 1, wherein the potassium is potassium citrate, and magnesium is magnesium citrate, with additional citric acid.

7. The method of claim 5, wherein the composition consists essentially of 20 meq potassium, 10 meq magnesium, and 37 meq citrate per dose.

8. The method of claim 5, wherein the composition consists of 20 meq of potassium, 10 meq magnesium, and 37 meq of citrate per dose.

9. The method of claim 1, wherein the thiazide is hydrochlorothiazide, or chlorthalidone.

10. The method of claim 9, wherein the thiazide is chlorthalidone.

11. The method of claim 1, wherein the patient has hypertension.

12. The method of claim 11, wherein the patient has essential hypertension.

13. The method of claim 1, wherein the composition is administered before the thiazide.

14. The method of claim 1, wherein the composition is administered after the thiazide.

15. The method of claim 1, wherein the composition is administered concurrently with the thiazide therapy.

16. The method of claim 1, wherein the patient is a human.

17. The method of claim 1, wherein the patient has been diagnosed with a metabolic disturbance.

18. The method of claim 1, wherein the metabolic disturbance is a thiazide-induced metabolic disturbance.

19. The method of claim 1, wherein the metabolic disturbance is insulin resistance, hypokalemia, activation of RAA-SNS, oxidative stress, dyslipidemia, increased FGF23 synthesis, or Mg depletion.

20. The method of claim 1, wherein said administration comprises oral administration.

21. The method of claim 1, wherein the composition is administered two, three, or four times per day.

22. The method of claim 1, wherein the composition is a powder or mixture.

23. The method of claim 22, wherein the powder is dissolved in water or aqueous media prior to oral ingestion.

24. The method of claim 22, wherein the powder or mixture is dissolved in water or aqueous media at a time period of one hour or less before oral ingestion.

25. The method of claim 22, wherein the powder or mixture is to be added to food before oral ingestion.

26. The method of claim 1, wherein the composition is in the form of a tablet.
